(19) United States
(12) Patent Application Publication
(10) Pub. No.: US 2008/0006275 A1
(43) Pub. Date: Jan. 10, 2008

(54) Composite masks and methods for positive airway pressure therapies

(76) Inventors: Steven Nickelson, Minneapolis, MN (US); Gregory Voss, Lakeville, MN (US); Charles Lehman, St. Louis Park, MN (US)

Correspondence Address: CYR & ASSOCIATES, P.A., 605 U.S. Highway 169, Suite 300, Plymouth, MN, 55441, US

(21) Appl. No.: 11/825,997

(22) Filed: Jul. 9, 2007

Related U.S. Application Data
(60) Provisional application No. 60/819,536, filed on Jul. 7, 2006.

(51) Int. Cl. A61M 16/06

(52) U.S. Cl. 128/206.21; 128/205.25

(57) ABSTRACT

Composite mask apparatus for providing positive pressure airway therapies are disclosed. The composite mask apparatus include an upper body and a lower body. The lower body is secured to a lower portion of the upper body. The upper body supports at least a portion of the lower body. A first branch of the upper body may be secured to a first tube of the lower body and a second branch of the upper body may be secured to a second tube of the lower body. An airway interface may be in fluid communication with a first lumen of the first tube and may also be in fluid communication with a second lumen of a second tube.

### CROSS-REFERENCE TO RELATED APPLICATIONS

This application claims the benefit under 35 U.S.C. §119(e) of U.S. Provisional Application Ser. No. 60/819,536 filed on Jul. 7, 2006, the disclosure of which is hereby incorporated by reference in its entirety.

### BACKGROUND OF THE INVENTION

1. Field of the Invention

The present inventions relate generally to respiratory devices and, more particular, to masks for positive airway pressure therapies.

2. Description of the Related Art

It is recognized that sleep apnea can be treated by use of positive airway pressure therapies. These therapies typically provide pressurized air at 4-20 cm water to a patient by way of the nose, and/or mouth. The therapies generally operate under the principle that positive air pressure administered to a user's airways during sleep may maintain airway patency during sleep. The use of continuous positive airway pressure therapies has become widespread for the treatment of chronic sleep apnea, chronic pulmonary obstruction and snoring. Many systems for administration of these therapies are now available. These systems generally include a source of pressurized air, tubing leading the source to the patient, and patient/tubing interfaces of various types.

One problem faced by manufacturers of positive airway pressure therapy devices is developing an adequate seal between the patient and the patient interface. Patient interfaces have commonly been configured as full nasal masks that surround the nose, full face mask that surround the nose and mouth, and masks that have outlets positionable in or about the nares. Each type has its own advantages and disadvantages for a given patient. A full nasal mask is generally made of a rigid material with a flexible “skirt” and must be firmly apposed to the face. Differences in facial topography may make the seal ineffective. Full face masks solve the problem of air leaks out of the mouth with some patients when using nasal masks, but again, topography of the face may make the seal ineffective and extra force is needed to maintain a seal against the face. Nares masks may have an advantage in that the point of contact between the nares, the area of the seal, is much reduced as are forces to maintain a seal.

Even though forces may be less to maintain a seal using certain mask configurations, the forces may need to be applied at a proper level and in the proper direction in order to maintain a good seal. Many mask configurations do not permit the precise control of forces without the addition of straps that pass over the face of a user. This may be particularly prevalent if the various stressed portions of the masks are themselves flexible and are connected to members other stressed portions that are also flexible. Although these designs may be comfortable, adequate forces may not be applied to maintain a seal without the addition of straps that pass over the face of a user. Such straps can be uncomfortable and may lead to a lack of compliance with the suggested therapy. Accordingly, a need exists for an apparatus and methods that permit the precise control of forces to position and seal a mask without the addition of additional straps.

If the various masks are made from solely from rigid materials, the seal position and seal forces against the user's face may not be maintained with slight changes in head position. The breaking of a seal can interrupt the therapy and can more generally disrupt the sleep of a user as the user is forced to reposition a mask. Accordingly, a need exists for apparatus and methods that provide for masks which may careful balance between adjustability, tension against the nares, and springiness to accommodate head movement needs to be available to allow ease of positioning, obtaining the minimum forces between the nares and the seal in order to maintain a seal during any head position and movement.

### SUMMARY OF THE INVENTIONS

Apparatus and methods in accordance with the present invention may resolve many of the needs and shortcomings discussed above and will provide additional improvements and advantages as will be recognized by those skilled in the art upon review of the present disclosure.

The present inventions provide composite mask having at least a first upper branch and a second upper branch secured to a first lower tube and a second lower tube, respectfully. In one aspect, the first upper branch may be secured within a lumen of a first lower lumen of the first lower tube. In another aspect, the second upper branch may be secured within a lumen of a second lower lumen of the second lower tube. In another aspect, the first upper branch may be secured over an outer surface of the first lower tube. In another aspect, the second upper branch may be secured over the outer surface of the second lower tube. In another aspect, an end of the first upper branch may be secured to and abutting an end of the first lower tube. In another aspect, an end of the second upper branch may be secured to and abutting an end of the second lower tube. The first upper branch defines a first upper branch passage. The second upper branch defines a second upper branch passage. An upper body may form the first upper branch and the second upper branch. The first lower tube and the second lower tube may be secured to or integral with a lower body. The first upper branch and the second upper branch may be formed from a material that is more rigid than the material of the lower body. The upper body may also be formed from a material that is more rigid than the material of the lower body. The lower body further including an airway interface. The airway interface may be in fluid communication with at least one of the first lower tube and the second lower tube. The airway interface configured to provide pressurized air through the nares of a user to permit the administration of positive airway pressure therapy to a user. The first upper branch configured to support at least a portion of the first lower tube. The second lower tube secured to the second upper branch with the second upper branch configured to support at least a portion of the second lower tube. In one aspect, at least one of the first upper branch and the second upper branch may transition in stiffness between an upper portion and a lower portion of at least one of the first upper branch and the first lower branch. In another aspect, at least one of the first upper branch and the second upper branch may include one or more slots extending along their length to confer a desired flexibility. In another aspect, at least one of the first upper branch and the second upper branch may include one or more grooves extending along their length to confer a desired flexibility. In another aspect, at least one of the first upper branch and the second upper branch may have increased flexibility in at least one plane of movement. In another aspect, at least one of the first upper branch and the second upper branch may have increased flexibility in one plane of movement.

In another aspect, the present inventions feature methods of providing positive airway pressure therapies to a user using the nasal mask. The composite mask in accordance with aspects of the present inventions is positioned over the airways of a user. Once positioned, pressurized air is provided through the composite mask to the airways of a patient. The user completes a sleep cycle with the positive airway pressure therapy having been administered through the composite mask.

Other features and advantages of the invention will become apparent from the following detailed description, and from the claims.

### BRIEF DESCRIPTION OF THE DRAWINGS

FIG. 1 is a perspective view of an embodiment of a gas supply apparatus in accordance with aspects of the present inventions fitted on a user with the gas supply apparatus including an embodiment of a composite mask according to the invention;

FIG. 2 is a perspective view of an embodiment of a composite mask in accordance with aspects of the present inventions;

FIG. 3 is a frontal view of an embodiment of a composite mask in accordance with aspects of the present inventions;

FIG. 4 is a rear view of an embodiment of a composite mask in accordance with aspects of the present inventions;

FIG. 5 is a perspective view of an embodiment of a composite mask in accordance with aspects of the present inventions having a portion of the composite mask cut away;

FIG. 6 is a perspective view of an embodiment of a supporting body for a composite mask in accordance with aspects of the present inventions;

FIG. 7 is an exploded view of an embodiment of a composite mask in accordance with aspects of the present invention with the supporting body separated from the lower tubes;

FIG. 8 is an exploded view of another embodiment of a composite mask in accordance with aspects of the present invention with the supporting body separated from the flexible body;

FIG. 9A is a frontal view of an embodiment of a supporting body for a composite mask in accordance with aspects of the present inventions;

FIG. 9B is a frontal view of another embodiment of a supporting body for a composite mask in accordance with aspects of the present inventions;

FIG. 9C is a side view of another embodiment of a supporting body for a composite mask in accordance with aspects of the present inventions;

FIG. 9D is a side view of another embodiment of a supporting body for a composite mask in accordance with aspects of the present inventions;

FIG. 10A is a partial view in longitudinal cross section illustrating an embodiment of a junction between the supporting body and a lower tube of a flexible body in accordance with aspects of the present inventions;

FIG. 10B is a partial view in longitudinal cross section illustrating another embodiment of a junction between the supporting body and a lower tube of a flexible body in accordance with aspects of the present inventions;

FIG. 10C is a partial view in longitudinal cross section illustrating another embodiment of a junction between the supporting body and a lower tube of a flexible body in accordance with aspects of the present inventions;

FIG. 10D is a partial view in longitudinal cross section illustrating another embodiment of a junction between the supporting body and a lower tube of a flexible body in accordance with aspects of the present inventions; and

FIG. 11 is a perspective view of a shaping wire.

All Figures are illustrated for ease of explanation of the basic teachings of the present invention only; the extensions of the Figures with respect to number, position, relationship and dimensions of the parts to form the preferred embodiment will be explained or will be within the skill of the art after the following description has been read and understood. Further, the exact dimensions and dimensional proportions to conform to specific force, weight, strength, and similar requirements will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings and utilized only to facilitate describing the illustrated embodiments. Similarly, when positional terms are used, the terms should be understood to reference the structures shown in the drawings as they will typically be utilized by a user being treated with an apparatus in accordance with one or more of the present inventions.

### DETAILED DESCRIPTION OF THE INVENTION

When used in this application, “appose” means to press one surface against another.

The present inventions include composite masks 18 configured for use in positive airway pressure therapies. Various embodiments of composite masks 18 and their components in accordance with aspects of the present inventions are generally illustrated throughout the figures for exemplary purposes.

Composite masks 18 in accordance with the present inventions are generally configured to communicate pressurized air from an air supply line 10 into the nares of a user. Composite masks 18 can include an upper body 120 secured to a lower body 140. The upper body 120 may include one or more of an upper support 122, a first upper branch 126 and a second upper branch 128. The lower body 140 may include at least one of a first lower tube 56 and a second lower tube 58. At least one of the first lower tube 56 and the second lower tube 58 are configured to direct pressurized air to one or more airway interfaces 65, such as a first naris interface 66 and a second naris interface 68 for example. The upper body 120 may be configured to interface with an air supply line 10 and to direct air into at least one of the first lower tube 56 and the second lower tube 58 of the lower body 140. In accordance with aspects of the present inventions, portions of the upper body 120 may function to support one or more portions of the lower body 140. In certain exemplary embodiments, the first upper branch 126 of the upper body 120 may support at least a portion of the first lower tube 56 of the lower body 140 and the second upper branch 128 of the upper body 120 may support at least a portion of the second lower tube 58 of the lower body 140.

As generally illustrated throughout the figures for exemplary purposes, the upper body 120 includes an upper support 122 with a first upper branch 126 and a second upper branch 128 extending from the upper support 122. The first upper branch 126 and the second upper branch 128 may define a first upper branch passage 136 and a second upper branch passage 138, respectively. In certain aspects, at least one of the first upper branch passage 136 and the second upper branch passage 138 are in fluid communication with an upper passage 124 defined by the upper support 122. The first upper branch 126 and the second upper branch 128 may include slots 132 or grooves 134 to confer the desired flexibility. The slots 132 or grooves 134 may extend into the outer surface of the upper body 120 and, in certain embodiments, may extend from the outer surface of the upper body 120 into one or more of the first upper branch passage 136 and the second upper branch passage 138. The wall thickness of the first upper branch 126 and the second upper branch 128 may also be varied along their lengths to confer the desired flexibility. In one aspect, the thickness of the first upper branch 126 and the second upper branch 128 may be tapered toward their ends. In other aspects, the material of the first upper branch 126 and the second upper branch 128 may be configured to be more rigid toward the upper support and less rigid toward the end in fluid communication with the first lower tube 56 and the second lower tube 58. As such, the first upper branch 126 and the second upper branch 128 may be configured to transition from stiff at an upper portion to more pliable near the lower portion.

The lower body 140 may include a first lower tube 56, a second lower tube 58, an airway interface 65. The first lower tube 56 may define a first lower lumen 166. The second lower tube 58 may define a second lower lumen 168. The airway interface 65 is in fluid communication with at least one of the first lower lumen 166 and the second lower lumen 168. The airway interface 65 may be configured to include at least one of a first naris interface 66 and a second naris interface 68. Each of the first naris interface 66 and the second naris interface 68 may be configured to engage a naris to permit the administration of positive airway pressure therapy. When present, the first naris interface 66 and the second naris interface 68 are in fluid communication with at least one of the first lumen 166 of the first lower tube 56 and the second lumen 168 of the second lower tube 58. In one aspect, the airway interface 65 may be secured to a lower collar 64. As particularly illustrated, the first naris interface 66 and the second naris interface 68 may be secured to a lower collar 64. The lower collar 64 may define one or more passages or chambers. The lower collar 64 may be formed from a rigid material or a flexible material similar or the same as the material of the material of the first lower tube 56 and the second lower tube 58. The airway interface 65 may be in fluid communication with the one or more passages or chambers of the lower collar 64. The first lower tube 56 and the second lower tube 58 may be secured to the lower collar 64 and may be in fluid communication with the one or more passages or chambers of the lower collar 64.

The lower body 140 can be secured to the upper body 120 to form a composite mask 18 in accordance with one or more aspects of the present inventions. The lower body 140 may be secured over at least a portion of the upper body 120. The first upper branch 126 may be secured to the first lower tube 56 and the second upper branch 128 may be secured to the second lower tube 58 to permit the flow of fluid through at least one of the first lower tube 56 and the second lower tube 58 to the airway interface 65. In one aspect, the first lower tube 56 may be secured over the first upper branch 126 and the second lower tube 58 may be secured over the second upper branch 128. In another aspect, the first lower tube 56 may be secured within the first upper branch 126 and the second lower tube 58 may be secured within the second upper branch 128. In another aspect, an upper end of the first lower tube 56 may be secured to a lower end of the first upper branch 126 and an upper end of the second lower tube 58 may be secured to a lower end of the second upper branch 128 either or both in an abutting type relationship.

The more rigid upper body 120 may be configured to provide mechanical support to various aspects of the more flexible lower body 140. The upper body 120 may be further or alternatively configured to maintain the integrity of at least a portion of the first lumen 166 of the first lower tube 56 and/or at least a portion of the second lumen 168 of the second lower tube 58. The upper body 120 may be further or alternatively configured to cooperate with an upper collar 28 or may include an integral upper collar 28 to secure the composite mask 18 to an air supply line 10 and/or a support 12. Typically, the upper body 120 is formed from one or more materials that are more rigid than the material or materials from which the first lower tube 56 and the second lower tube 58 are formed. Upper body 120 may be formed from a variety of relatively rigid materials including for example polymethyl methacrylate, polystyrene, polycarbonate or other rigid materials. The rigidity of the material is generally selected to permit the upper body 120 to not substantially deform while maintaining contact with a user during the administration of a positive airway pressure therapy. By contrast, the lower body 140 may be formed from a variety of relatively flexible and/or deformable materials including for example silicone rubber or a variety of other rubber or plastics materials. The material is generally selected to permit the shaping of the lower body 140 and/or to comfortably contact the skin of a user. The upper support 122 may define an upper passage 124. In one aspect, the upper passage 124 may be in fluid communication with at least one of the first upper passage 136 and the second upper passage 138. The first upper passage 136 and the second upper passage 138 may be in fluid communication with the first lumen 166 of the first lower tube 56 and the second lumen 168 of the second lower tube 58, respectively. Accordingly, the first upper branch lumen 136 may be in fluid communication with the first naris interface 66 through the first lumen 166 of the first lower tube 56 and the second upper branch lumen 138 may be in fluid communication with the second naris interface 68 through the second lumen 168 of the second lower tube 58.

The lower body 140 is generally configured to direct a fluid into the nares of a user. At least portions of the lower body 140 are typically formed from a flexible material or flexible materials to permit the composite mask 18 to be fitted to a patient. The lower body 140 typically includes a first lower tube 56, a second lower tube 58, a first naris interface 66 and a second naris interface 68. The lower body 140 may also include an upper sleeve 142. The upper sleeve 142 of the lower body 140 may extend over at least a portion of the upper support 122 of the upper body 120. A shaping wire 40 may be provided to at least assist in retaining a desired shape for proper fitment. In one aspect, the first lower tube 56 and the second lower tube 58 are formed from a flexible material and a shaping wire 40 extends along at least a portion of each of their length. The lower body 140 may also include a lower collar 64.

Composite masks 18 in accordance with the present inventions may be used in combination with a variety of caps, harnesses or other support structures 12. Typically, the support structures 12 lead an air supply line 10 to a position above the nose. The air supply line 10 may terminate in a connector and/or opening to which the composite mask 18 can be attached, removably or otherwise. When attached, the composite mask 18 generally extends from that connector towards the nares.

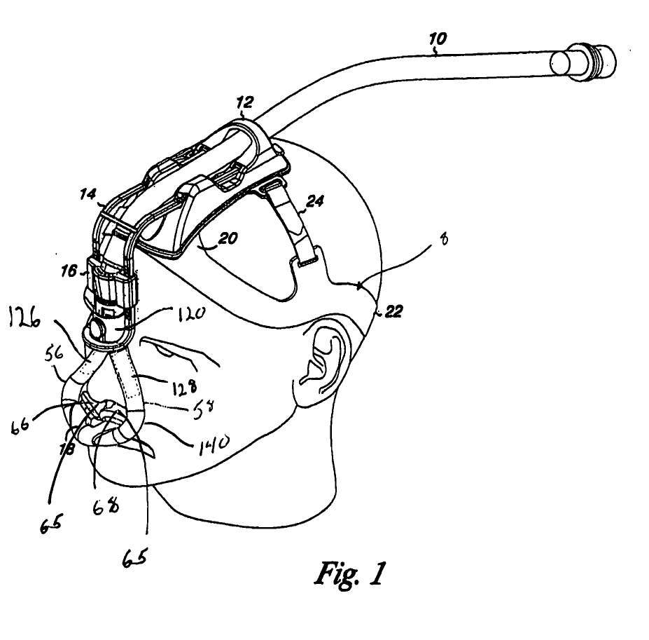

An exemplary composite mask 18 secured to a support structure is particularly illustrated in FIG. 1. As illustrated, composite mask 18 and associated support structure 12 is configured for communicating a gas to the nose of a patient to administer a continuous positive air pressure therapy for treatment of snoring and/or sleep apnea. The apparatus of FIG. 1 includes a support 12. The support 12 is typically formed from a rigid or semi-rigid molded plastic material. The support 12 may have a length greater than its width and having a concave lower surface profiled so that the support can rest on the top of the head of a user. The support 12 may include an intermediate layer of deformable material between the rigid or semi-rigid molded plastic material and the user's head. The support 12 may be sized to extend partway across the head and to extend between the top of the head and a region of the forehead. Headgear 8 is shown in the form of an open cap for holding the support 12 stably on the head and includes a circumferential stabilizing band 22 for fitting around the head between a lower back region of the head and a region of the forehead. The headgear 8 may also be provided with anterior attachment 20 to the support 12, and with lateral attachments to the support 12 via bands 24. These bands 24 may assist in stably holding the support 12 on the head when the circumferential band is fitted to the head. A mask holder 14 may be secured to the support 12. The mask holder may also be formed from a rigid or semi-rigid material e.g. of glass-filled nylon that may extend forwardly from and may be stably carried by the support 12. The mask holder 14 may be sized and shaped to extend forward beyond the face and a region of the forehead and then downward towards the nose. A lower portion of the mask holder 14 may be provided with connector carrier 16 which may include a plenum. An upper connector may lead to the plenum. A lower plug connector (not shown) may lead from the plenum and face downwards generally parallel to the vertical direction of the face. An air supply line 10 may passes through or about the support 12 and the mask holder 14 to the upper part of the connector carrier 16 where it joins the upper connector. A composite mask 18 in accordance with one or more of the present inventions may be attached to the lower plug connector and depend from the connector carrier 16.

A rear upper region of the mask holder 14 may be adjustable in an anterior-posterior direction relative to support 12 for adjustably positioning the connector carrier 16 towards and away from the face, for which purpose the mask holder 14 may be toothed to cooperate with a bi-directional clicker or ratchet mechanism (not shown) forming part of the support 12. Thereby, the mask holder 14 may be moved to a desired position and may then be retained in the selected position until it is positively readjusted. Similarly, the connector carrier 16 may be adjustable upward or downward relative to the face on the lower anterior region of mask holder 14 which may also be toothed, the connector carrier 16 may include a second bi-directional clicker or ratchet mechanism (not shown) by which it can be adjusted to a selected vertical position on the mask holder 14, and may be thereafter retained in its selected vertical position until readjusted.

The upper collar 28 of the composite mask 18 may be connectable to the plug connector or other connector device of the connector carrier 16. The orifice 26 may be attached via the connector to the plenum and thence to a blower or other air source to confer a positive airway pressure therapy. The upper collar 28 may be of any size to accommodate attachment to any plenum and air supply. Although the composite mask 18 may be made in a range of sizes for different patients, in a typical size it is of overall height about 115 millimeters, overall width about 66 millimeters and internal volume about 20-50 milliliters in certain embodiments.

When assembled, the composite mask 18 may be devoid of openings other than an upper orifice 26 and nares tubes described below. In one aspect, internal volume of the composite mask 18 may be only a small fraction of the volume of air inhaled or exhaled at each breath, which is typically about 500 ml, so that no apertures for exhaled air need be formed in composite mask 18. Instead, upwardly facing apertures for escape of excess air flowing from the blower through the air supply line 10 and for escape of exhaled air can be provided at the connector carrier 16, the apertures opening through the upper surface of connector carrier 16 immediately in front of the obliquely facing hose connector.

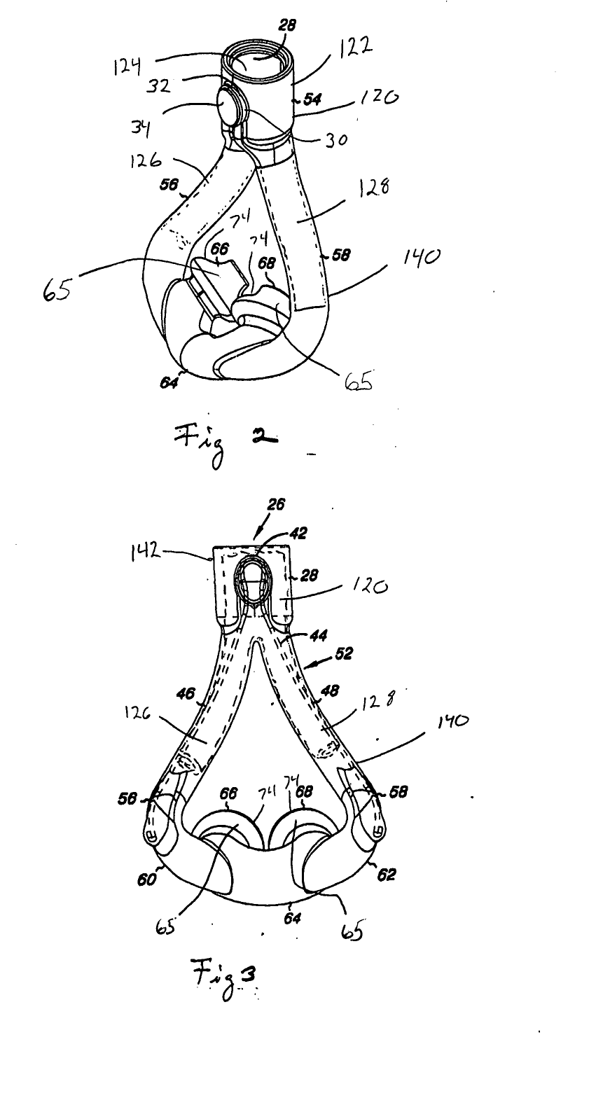

As particularly illustrated in FIGS. 2 and 3, the lower body 140 is formed from a soft flexible material which extends over a portion of upper body 120 in FIG. 2 and substantially covers upper body 120 in FIG. 3. This flexible material may be a medical grade silicone rubber and may be formed into a triangular shape defining an upper region or space 54 that fits over and surrounds the connector 28. As illustrated, the upper support 122 may bifurcate to define the first upper branch 126 (shown in phantom) and the second lower branch 128 (shown in phantom). The first upper branch 126 and the second upper branch 128 may be laterally spaced and extend downward. In one aspect, they may form a triangular space which may be particularly configured to receive the nose of a patient. The first upper branch 126 and the second upper branch 128 as well as the first lower tube 56 and second lower tube 58 may be of generally circular, elliptical, oval profile or otherwise shaped. When elliptical or oval, the first upper branch 126 and the second lower branch 128 and the first lower tube 56 and second lower tube 58 may have an aspect ratio of about 1.1 to 1.9 in certain embodiments. With an anterior-posterior major inner dimension for an adult patient of typically of about 8 to 12 millimeters in certain embodiments and with a transverse minor dimension typically of about 5 to 9 millimeters in those embodiments, the oval or elliptical shape with these proportions may provide dimensions conferring the designed physical characteristics while being relatively unobtrusive to a user's vision. As illustrated, a shaping wire 40 is integrally molded into the tubes 56, 58. For exemplary purposes, the shaping wire is shown positioned near the outside (front) edges of the tubes 56, 58. The shaping wire 40 may provide mild apposing force to hold flanges 74 of the outlet tubes 66, 68 (described below) against the nares. The shape of the lower body may also be adjusted slightly to accommodate differences in facial topology by bending the shaping wire 40. In the illustrated structure, the limbs 46, 48 of the shaping wire 40 are molded into ribbed pockets which extend along the front of each tube 56, 58.

When viewed in profile, the tubes 56, 58, 126 and 128 may be curved in an anterior direction, with the steepness of curvature increasing progressively with distance from region 54. Below the nose, and below the ends of wire limbs 46, 48, the tubes 56, 58 merge into relatively short transition regions 60, 62 which curve obliquely inwardly and in turn merge into a generally U-shaped lowermost region 64 or collar which may be slightly larger than tubes 56, 58, which may be approximately 12 to 18 millimeters across. The lower collar 64 may support outlet tubes 66, 68. The outlet tubes 66, 68 may be directed obliquely rearwardly and upwardly at an angle of about 20 to 40 degrees in certain embodiments as viewed in FIG. 4 so as to fit into the anterior nares of the patient.

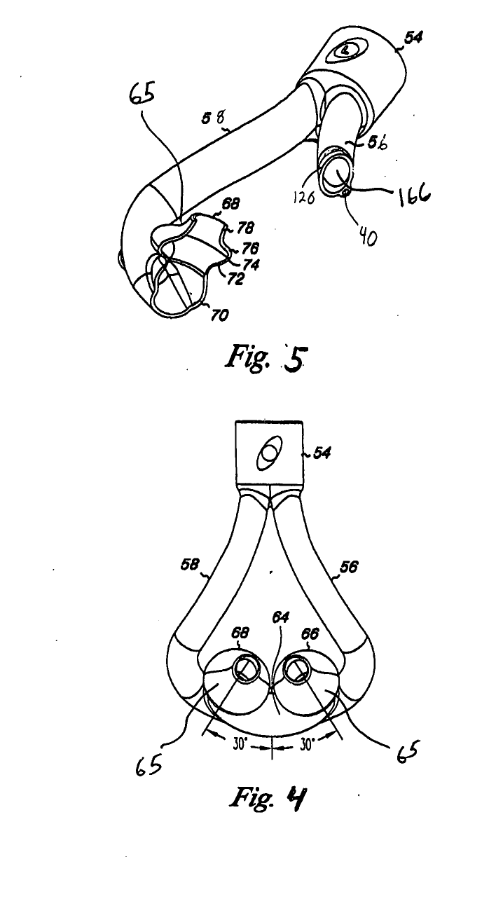

As can be seen in FIGS. 4 and 5, the flanges 74 of the two outlet tubes 66, 68 and the two distal regions 78 may both have an oval shape. With the anterior-posterior direction as shown, the distal regions 78 may be about 10 millimeters by 7 millimeters. In use, the distal regions 78 become inserted into the anterior nares of the patient, with the flanges 74 acting as seals to prevent air leakage.

In FIG. 5, the tubes 56 and 126 are shown partially cut-away to reveal a portion of shaping wire 40 extending from tube 126 and to reveal the oval shape of the tubes 56. Outlet tube 68 is illustrated in with a relatively thick base region 70 leading to flange 74 that may be defined by relatively thin anterior region 72 and thicker posterior region 76. The posterior region 76 flares into reduced diameter distal region 78.

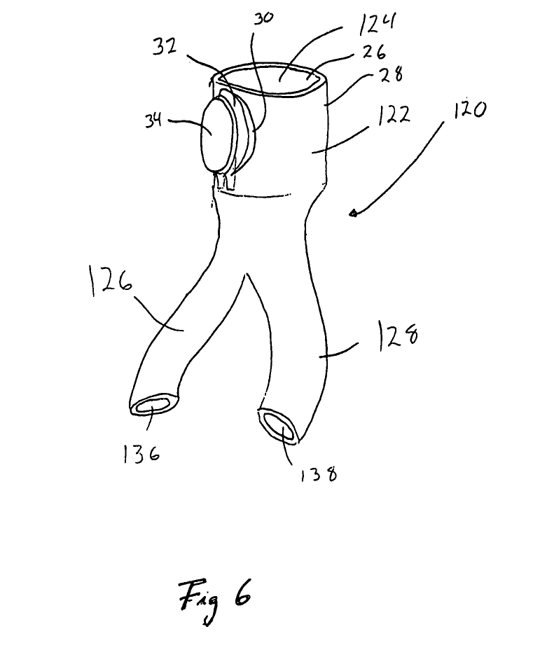

FIG. 6 shows an upper body 120 having an integral connector 28. The illustrated connector 28 is integral with an upper support 122 of upper body 120. Upper support 122 is configured as a tube defining an upper lumen 124. The upper body bifurcates into a first upper branch 126 and a second upper branch 128. The first upper branch 126 defines a first upper branch lumen 136 and the second upper branch 128 defines a second upper branch lumen 138. The illustrated connector 28 defines an orifice 26. The connector 28 may be located at the top of upper body 120 and/or composite mask 18. The connector 28 may be configured to secure the composite mask 18 to the connector carrier 16. The inner surface of the connector 28 may be formed with a detent to permit the connector to make a push-and-turn fit with formations on the plug connector which depends from the connector carriage 16. The outer surface of the connector 28 may be formed with a boss 30 leading via a recessed web region 32 to button 34. The boss 30 leading via a recessed web region 32 to button 34 may be configured to secure a portion of the shaping wire 20 to the upper body 120.

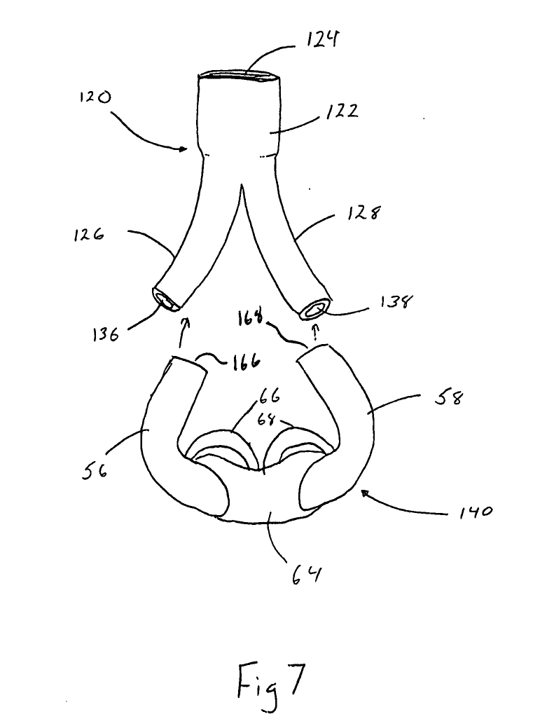

FIGS. 7 and 8 illustrate exploded view of an upper body 120 separated from a lower support 140. As illustrated in FIG. 7, the upper body 120 and the lower body 140 overlap over the first upper branch 126 and second upper branch 128 and the first lower tube 56 and second lower tube 58, respectively. As illustrated in FIG. 8, the upper body 120 and the lower body 140 overlap over substantially the entire length of the upper body 120 from the upper aspect of the upper support 122 through the first upper branch 126 and second upper branch 128 and over a portion of the length of the lower body 140 from the upper sleeve to the first lower tube 56 and second lower tube 58.

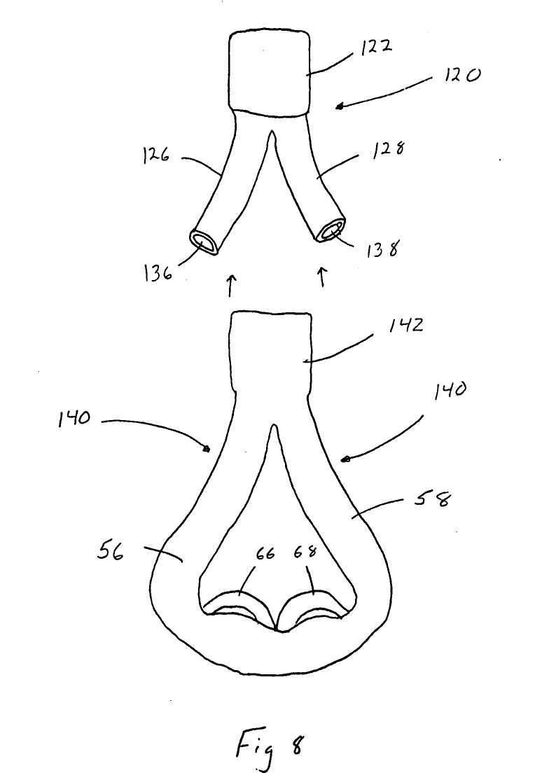

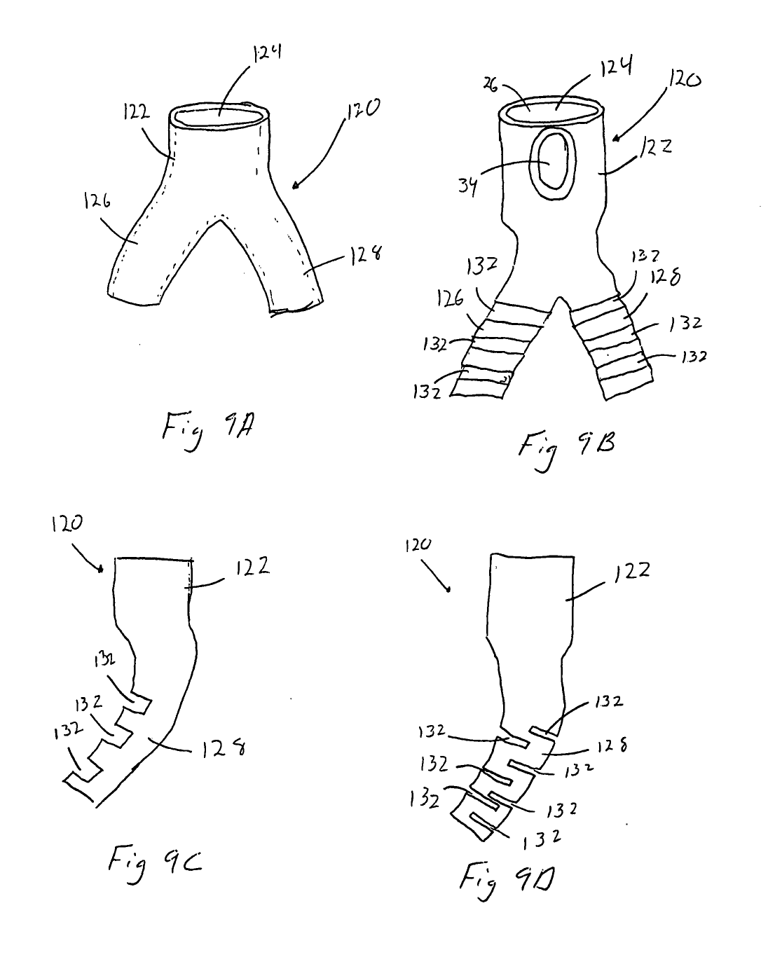

FIGS. 9A to 9D illustrate various configurations for aspects of the upper supports 120. FIG. 9A illustrates front view of an upper body 120 having the upper support 122 configured as a tube and having the first upper branch 126 and second upper branch 128 in a solid configuration. FIG. 9B illustrates front view of an upper body 120 having the upper support 122 configured as a tube and having the first upper branch 126 and second upper branch 128 including a spirally cut slot 132 extending from a location inferior to the branching point to the lower end of the first upper branch 126 and second upper branch 128. The spirally cut slot 132 may be configured to confer the desired flexibility characteristics on the first upper branch 126 and second upper branch 128 of the upper body 120. FIG. 9C illustrates side view of an upper body 120 having the upper support 122 configured as a tube and having at least the second upper branch 128 including a plurality of slots 132 cut in an anterior surface and extending from a location inferior to the branching point to the lower end of the first upper branch 126 and second upper branch 128. The anteriorly positioned slots 132 may be configured to confer the desired flexibility characteristics on at least the second upper branch 128 of the upper body 120. FIG. 9D illustrates side view of an upper body 120 having the upper support 122 configured as a tube and having at least the second upper branch 128 including a plurality of slots 132 cut into both its anterior and posterior surfaces and extending from a location inferior to the branching point to the lower end of the first upper branch 126 and second upper branch 128. The anteriorly and posteriorly positioned slots 132 may be configured to confer the desired flexibility characteristics on at least the second upper branch 128 of the upper body 120.

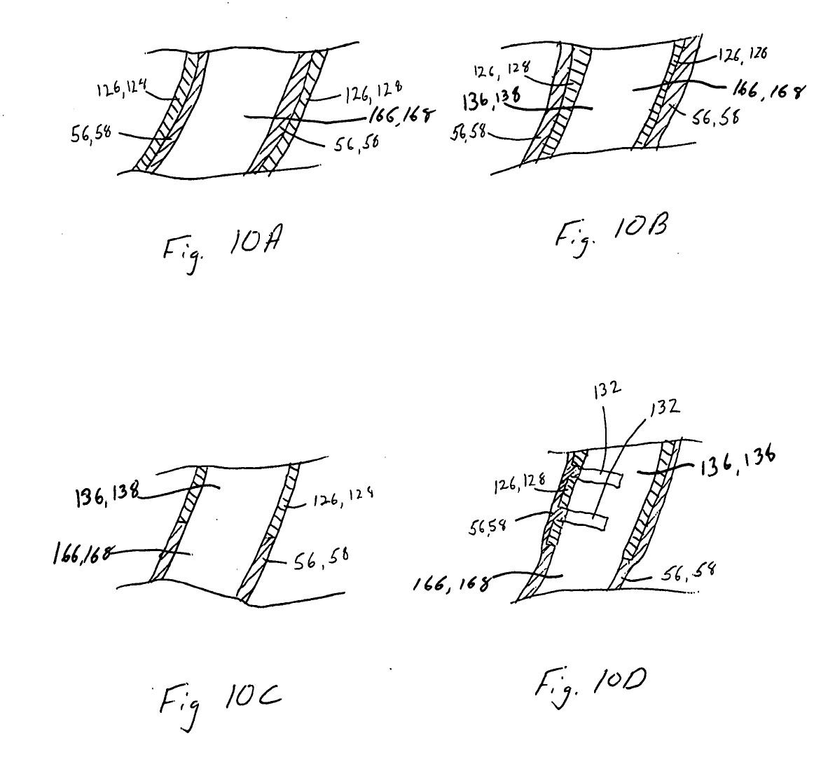

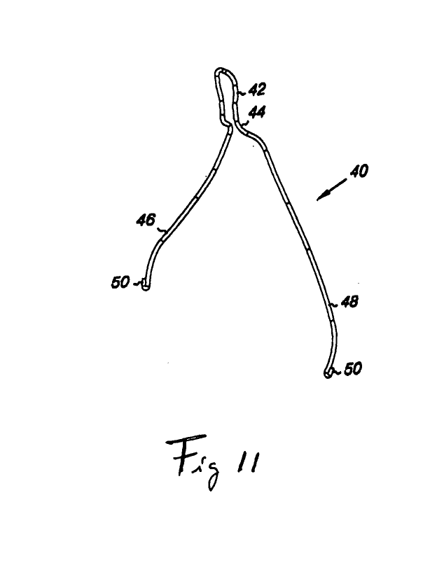

FIGS. 10A to 10C illustrates partial cross sectional side views of exemplary intersections and/or overlaps of the first upper branch 126 and second upper branch 128 and the first lower tube 56 and second lower tube 58. FIG. 10A illustrates the upper branch 126, 128 secured over the lower tube 56, 58. FIG. 10B illustrates the upper branch 126, 128 secured within the lower tube 56, 58. FIG. 10C illustrates a lower end of the upper branch 126, 128 secured to an upper end the lower tube 56, 58 in an abutting configuration. FIG. 10D illustrates the upper branch 126, 128 secured within the lower tube 56, 58 and the material of the lower tubes 56, 58 extending into slots 132 of the upper branch 126, 128 to fill the gaps defined by the slots 132 and to form a substantially smooth unbroken inner surface for passage 166,168 to reduce turbulence in the fluid flow through the passage 166, 168.

A first length of shaping wire 46 and a second length of shaping wire 48 may extend through or be otherwise secured to aspects of the first lower tube 56 and/or the second lower tube 58 to permit the proper fitting of the airway interface 65. As particularly illustrated in FIG. 11 for exemplary purposes, a single shaping wire 40 including both a first length of shaping wire 46 and a second length of shaping wire 48 may be provided of generally inverted U-shape. The U-shape may include upper relatively narrow loop region 42 for anchoring in load-transmitting relationship over web region 32 between boss 30 and button 34 so that the web region 32 can provide support to shaping wire 40 against downward load arising at the nares. The length of the loop region 42, the length of the web region 32 including its downward extension pieces stabilize the position of shaping wire 40 against relative movement in a medial-lateral direction and boss 30 and button 34 stabilize it against movement in an anterior-posterior direction. The loop region 42 leads to relatively short divergent transition region 44 which may be offset inwardly of the connector 28 or rearwardly relative to the face and in turn leads to relatively lengths of shaping wire 46, 48 that terminate at short regions 50 where the cut ends of the lengths of shaping wire 46, 48 are returned. The wires can be of any material that may be easily deformable but that retains its shape unless deliberately deformed. The shaping wire 40 is may be formed from a metal such as for example brass or steel. In certain embodiments, the shaping wire may be formed stainless steel which is nickel-plated. Each side of the shaping wire 40 may be of length between about 20 and 100 millimeters depending upon the particular configuration.

As explained above, the composite mask 18 may secured to and can extend from a support 12 to stably position the composite mask 18 about the nose and to be connected to the support by a rigid connector at the top of the seal so that the support can provide a reaction to forces on the mask. In positive airway pressure therapies, air from a blower may be supplied to the seal 18 in excess of breathing requirements so that the pressure within the mask is positive, falling somewhat during inhalation and rising somewhat during exhalation.

The composite mask 18 may be subject to pressure of gas in the nares which produces loads acting obliquely forwardly and downwardly at an angle of about 20 to 40 degrees, along the axes of the tubes 66, 68 in certain embodiments. The downward components of these loads are reacted by the connector 28, which may be mechanically connected to a connector of the plenum. Compressive load between the seal and the nares, and the forward component of the load can deflect the relatively thin deformable region 72 of each outlet tube to facilitate fitting of the mask to the face. The U-shaped lower collar 64 can react load along the axes of the outlet tubes with relatively little deflection, and the transition regions 62, 64 though also composed of silicone rubber or other deformable material only without shaping wires also undergo relatively little deflection. The transition regions apply the load predominantly to wire limbs 46, 48, which stiffen the tubes 56, 58, and the load may be reacted at connector 28 at button 34. The combination of the stiffness of the wire support 40 with the stiffness of the silicone rubber or other material of tubes 56, 58 enables the flanges 74 to be held apposed to the anterior nares and to resist displacement from the nares even when the patient exhales strongly. The distal regions 78 remain in position in the nares and are remarkably resistant to displacement from their correct position even on vigorous exhalation intended to produce this result. Despite the construction in flexible materials such as silicone rubber and relatively thin shaping wire, embodiments of the present mask can provide an unexpectedly stable interface between the nares and overlying portions of a patient interface.

The body of the seal may be molded in one piece from silicone rubber or other deformable rubber or plastics material, with the shaping wire 40 introduced into the mold so that it becomes embedded in the material of the body. The region 64 may be formed with a slotted opening to give access to forming tools during the molding process, the slot being heat-sealed after molding. The connector 28 may then be fitted to upper region 54 of the mask body. For that purpose, region 54 as molded may be provided with an oval aperture through which button 34 appears. In order to fit the connector 28, the region 54 may be pulled downwardly away from the looped region 42, after which web region 32 is introduced between the uppermost part of limbs 46, 48 and the connector may be engaged with region 42 to anchor the top loop 42 of the shaping wire. The region 54 may be then folded back over the connector 28, after which additional silicone may be coated over the opening in the region 54 to conceal the button 34 and cured.

The foregoing discussion discloses and describes merely exemplary embodiments of the present inventions. Upon review of the specification, one skilled in the art will readily recognize from such discussion, and from the accompanying drawings and claims, that various changes, modifications and variations can be made without departing from the spirit and scope of the inventions as defined in the following claims.

What is claimed is:

1. A composite mask, comprising:
a first upper branch, the first upper branch defining a first upper branch passage;
a second upper branch, the second upper branch defining a second upper branch passage; and
a lower body including a first lower tube, a second lower tube, and an airway interface, the first lower tube secured to the first upper branch with the first upper branch configured to support at least a portion of the first lower tube, the second lower tube secured to the second upper branch with the second upper branch configured to support at least a portion of the second lower tube, the airway interface in fluid communication with at least one of the first lower tube and the second lower tube, and the airway interface configured to provide pressurized air through the nares of a user to permit the administration of positive airway pressure therapy to a user.

2. A composite mask as in claim 1, further comprising the first upper branch and the second upper branch formed from a material that is more rigid than the material of the lower body.

3. A composite mask as in claim 1, further comprising an upper body forming the first upper branch and the second upper branch.

4. A composite mask as in claim 3, further comprising the upper body formed from a material that is more rigid than the material of the lower body.

5. A composite mask as in claim 1, further comprising at least one of the first upper branch and the second upper branch transitioning in stiffness between an upper portion and a lower portion of at least one of the first upper branch and the first lower branch.

6. A composite mask as in claim 1, further comprising at least one of the first upper branch and the second upper branch including one or more slots extending along their length to confer a desired flexibility.

7. A composite mask as in claim 1, further comprising at least one of the first upper branch and the second upper branch including one or more grooves extending along their length to confer a desired flexibility.

8. A composite mask as in claim 1, further comprising at least one of the first upper branch and the second upper branch having increased flexibility in at least one plane of movement.

9. A composite mask as in claim 1, further comprising at least one of the first upper branch and the second upper branch having increased flexibility in one plane of movement.

10. A composite mask as in claim 1, further comprising the first upper branch secured within a lumen of a first lower lumen of the first lower tube.

11. A composite mask as in claim 10, further comprising the second upper branch secured within a lumen of a second lower lumen of the second lower tube.

12. A composite mask as in claim 1, further comprising the first upper branch secured over an outer surface of the first lower tube.

13. A composite mask as in claim 12, further comprising the second upper branch secured over the outer surface of the second lower tube.

14. A composite mask as in claim 1, further comprising an end of the first upper branch secured to and abutting an end of the first lower tube.

15. A composite mask as in claim 14, further comprising an end of the second upper branch secured to and abutting an end of the second lower tube.
